(19) United States
(12) Patent Application Publication
(10) Pub. No.: US 2026/0263823 A1
(43) Pub. Date: Sep. 10, 2026

(54) AUTOMATED EXTERNAL DEFIBRILLATOR WITH SUPPLEMENTAL POWER BANK

(71) Applicant: Avive Solutions, Inc., Brisbane, CA (US)

(72) Inventors: Andreea F. MARTIN, Burlingame, CA (US); Priscilla M. AGOSTO, San Francisco, CA (US); Dev PATEL, Foster City, CA (US); Gregory M. FENEIS, Redwood City, CA (US); Jimy PESIN, San Mateo, CA (US)

(21) Appl. No.: 19/557,512

(22) Filed: Mar. 5, 2026

Related U.S. Application Data
(60) Provisional application No. 63/768,376, filed on Mar. 7, 2025.

(51) Int. Cl. A61N 1/39; H01M 10/44; H01M 50/247; H01M 50/256; H02J 7/34; H02J 7/60; H02J 7/80; H02J 7/90; H02J 7/94

(52) U.S. Cl. CPC A61N 1/3975; A61N 1/3904; A61N 1/3931; A61N 1/3968; A61N 1/3993; H01M 10/44; H01M 50/247; H01M 50/256; H02J 7/342; H02J 7/685; H02J 7/80; H02J 7/94; H02J 7/971; H02J 2207/30

(57) ABSTRACT

Systems and methods are described that help manage supplemental power banks used to recharge AEDs. In one aspect, a power management controller is configured to periodically automatically enter an active mode thereby making power available from the power bank to the AED's power input interface, and thereafter automatically enter a deactivated mode such that power is not available to the AED until the active mode is reentered. The power management controller may be incorporated into a power delivery accessory, the power bank or the AED. In another aspect, a power delivery accessory includes a parasitic charging circuit that charges an energy storage device that powers the accessory.

### CROSS REFERENCE TO RELATED APPLICATIONS

This application claims priority of Provisional Application No. 63/768,376, filed Mar. 7, 2025, which is incorporated herein by reference in its entirety.

### FIELD

The present disclosure pertains to power management systems for extending the shelf life of automated external defibrillators (AEDs) and related devices using supplemental power banks.

### BACKGROUND

Automated External Defibrillators (AEDs) are life-saving medical devices designed to respond to sudden cardiac arrest (SCA), one of the leading causes of death worldwide. AEDs are deployed in both public and private spaces, including offices, schools, airports, fitness centers and many other locations. Once deployed, AEDs remain on standby for extended periods. These devices must be operational whenever an emergency arises, as their availability and functionality can mean the difference between life and death.

To ensure operational readiness, AEDs typically perform regular self-tests and have visual indicators, such as a status light, to reflect their operational status. Although indicator lights can be effective, they require periodic human monitoring, which is not always feasible. AEDs deployed in unattended locations or areas where their operational status is infrequently checked risk being non-functional when needed. For example, one recent study found that nearly one-fifth of the registered and publicly accessible AEDs in the study area were non-functional. Functionality of Registered Automated External Defibrillators, Jespersen et. al, Resuscitation 176 (2022) 58-63.

To help address these issues, some modern AEDs incorporate connectivity features, allowing them to communicate their operational status to a central server. These “connected AEDs” periodically transmit data, such as battery levels, self-test results, and overall operational health, to ensure proactive identification of maintenance needs. Connectivity significantly enhances the reliability and accountability of AEDs by reducing dependence on manual checks.

However, connected AEDs introduce unique challenges, particularly with respect to power management. Frequent check-ins, self-tests, and communication processes consume power, which can deplete the device's battery more rapidly than might otherwise occur. For AEDs that do not have access to continuous power sources for recharging, these demands pose a significant barrier to maintaining operational readiness over a multi-year lifetime.

Another trend for AEDs is reducing their size and weight to increase portability. This leads to designs with smaller and/or rechargeable batteries. Smaller batteries that are not rechargeable tend to need to be replaced more frequently. Rechargeable batteries can work well when power is available at their storage location but require more frequent attention if they are kept at a location that does not have power available for recharging.

As a result, there is a need for improved power management systems and techniques for AEDs, especially for smaller and/or connected AEDs. Such advancements must address the competing requirements of extended wireless connectivity, cost efficiency, and ensuring that the AEDs are able to maintain operational status and connectivity throughout their lifetime.

### SUMMARY

A variety of systems are methods are described that help manage supplemental power banks used to recharge AEDs. In one aspect an automated external defibrillator (AED) system includes an AED having at least one rechargeable battery, a supplemental power bank suitable for making power available to the AED, and a power delivery accessory. The power delivery accessory controls the delivery of power from the supplemental power bank to the AED's power input interface. The power delivery accessory includes a power management controller. The power management controller is configured to cause the power delivery accessory to (i) periodically automatically enter an active mode thereby making power available from the supplemental power bank to the AED's power input interface, and (ii) thereafter automatically enter a deactivated mode such that power is not available to the AED's power input interface until the active mode is reentered.

In some embodiments, the power management controller is configured to directly or indirectly monitor a current supplied to the AED's power input interface while the power delivery device is in the active mode. The power management controller if further configured to deactivate the supplemental power bank based at least in part on a determination that the supplied current does not exceed a designated threshold.

In some embodiments, the power management controller is configured to activate the power delivery accessory no more often than once a day.

In some embodiments, the power delivery accessory takes the form of a dongle that includes a first connector for receiving power from the supplemental power supply and a charging interface for delivering power to the AED's power input interface. In some embodiments, the charging interface is selected from the group consisting of a second connector, a USB cable, a power cable and a wireless charger.

In some embodiments, the power delivery accessory includes an output switch controlled by the power management controller. When the output switch is turned on, power is made available to the AED's power input interface. When the output switch is turned off, power is not available to the AED's power input interface.

In some embodiments, the power management controller causes the supplemental power bank to be active less than 5% of the time, and more preferably, less than 1% of the time.

In some embodiments, the power management controller is configured to enter the active mode after a designated period of time has passed.

In some embodiments, the power management controller is further configured to detect new connections of the power delivery accessory to the AED and new connections of the power delivery accessory to the supplemental power bank. The power management controller causes the power delivery accessory to enter the active mode when a new connection is detected that results in the power delivery accessory being connected to both the AED and the supplemental power bank.

In some embodiments, the supplemental power bank has an output connector through which power suitable for charging the AED is made available to the power delivery accessory, and the power delivery accessory includes a disconnect spoofing circuit. When the disconnect spoofing circuit is set to a first state, it causes the supplemental power bank to deactivate such that power suitable for charging the AED is not made available to the power delivery accessory. When the disconnect spoofing circuit is set to a second state, it causes the supplemental power bank to activate such that power suitable for charging the AED is made available to the power delivery accessory through the output connector.

In some embodiments, the output connector of the supplemental power bank includes at least one power pin and at least one control pin,. Power suitable for charging the AED is made available via the power pin. The power delivery accessory includes a parasitic charging circuit and an energy storage device suitable for powering the power delivery accessory. The parasitic charging circuit utilizes power from the control pin to parasitically charge the energy storage device of the power delivery accessory. In some embodiments, the energy storage device of the power delivery accessory is parasitically charged while the supplemental power bank is deactivated. In some embodiments, the energy storage device of the power delivery accessory is also parasitically charged while the supplemental power bank is activated. In some embodiments, the output connector is a USB connector, and the control pin is a first configuration control pin of the USB connector.

In some embodiments, a carrying case is provided. The carrying case is configured to store the AED, the power accessory and the supplemental power bank. Thus, the carrying case facilitates charging the AED while stored. In some embodiments, the carrying case includes an AED support insert having a bracket that holds the AED and the supplemental power bank is positioned below the AED support insert on the opposite side of the AED support insert as the AED. In some embodiments, the carrying case includes a window that exposes a display screen of the AED such that the display screen is externally visible when the AED is stored in the carrying case. In some embodiments the carrying case further comprises a light pipe configured to make light from an indicator light on the AED more visible from outside of the carrying case when the AED is stored in the carrying case.

In another aspect, the supplemental power bank includes a battery and a power management controller. The supplemental power bank's power management controller is configured to periodically automatically activate the supplemental power bank to thereby make power available from the supplemental power bank whereby power from the supplemental power bank is made available to the AED's power input interface. The power management controller also automatically deactivates the supplemental power bank such that power is not available from the supplemental power bank until the supplemental power bank is next reactivated.

In some embodiments, the power management controller is configured to activate the supplemental power bank after a designated period of time has passed. The frequency of the activation may vary widely. By way of example, in some embodiments, the supplemental power bank is activated no more often than once a day.

In some embodiments, power from the supplemental power bank is made available to the AED via a power delivery accessory selected from the group consisting of a USB cable, a power cable, and a wireless charger.

In some embodiments, the supplemental power bank includes an output switch controlled by the power management controller. When the output switch is turned on, power is made available from the supplemental power bank whereby the supplemental power bank is active. When the outputs switch is turned off, power is not made available from the supplemental power bank whereby the supplemental power bank is deactivated.

In some embodiments, the power management controller causes the supplemental power bank to be active less than 5% of the time, and more preferably, less than 1% of the time.

In some embodiments, the AED includes a battery charging circuitry suitable for controlling charging of the at least one rechargeable battery using power received through the power input interface.

In another aspect, a device is coupled to a power bank having a first connector that includes a first power pin and a first control pin. The power bank has an active state and a deactivated state. When the power bank is in the active state, power is made available via the power pin. When the power bank is in the deactivated state, power is not made available via the power pin. The device includes a second connector, an energy storage unit and a parasitic charging circuit. The second connector couples to the first connector of the power bank and includes a second power pin that connects to first power pin and a second control pin that connects to the first control pin. The parasitic charging circuit is configured such that when the power bank is in the deactivated state, the parasitic charging circuit utilizes power received via the second control pin to parasitically charge the energy storage unit of the device.

In some embodiments, the energy storage device of the power delivery accessory is also parasitically charged while the supplemental power bank is in the active state.

In some embodiments, the first and second connectors are USB connectors, and the first and second control pins are configuration control pins of the respective first and second USB connectors.

In some embodiments, the device further comprising a disconnect spoofing circuit. Setting the disconnect spoofing circuit to a first state with the power bank connected causes the power bank to deactivate such that power is not made available to device through the first and second power pins. Setting the disconnect spoofing circuit to a second state with the power bank connected causes the power bank to activate such that power is made available to the device through the first and second power pins.

In another aspect, an automated external defibrillator system includes an AED, a supplemental power pack and a power delivery controller. The power delivery controller controls power delivery from the supplemental power bank to the AED. The power delivery controller is configured to at least sometimes toggle the power delivered to the AED in a predetermined sequence to identify a type of the supplemental power bank to the AED.

In some embodiments, the power delivery controller is in either the supplemental power bank or a power delivery accessory electrically connected between the supplemental power bank and the AED.

### BRIEF DESCRIPTION OF THE DRAWINGS

The invention and the advantages thereof, may best be understood by reference to the following description taken in conjunction with the accompanying drawings in which:

FIG. 1 is a diagrammatic representation of an AED with a supplementary power bank in accordance with one embodiment.

FIG. 2 is an electrical block diagram illustrating representative control electronics for the supplemental power bank shown in FIG. 1.

FIG. 3 is a flow chart illustrating a recharging protocol in accordance with one embodiment.

FIG. 4 is a diagrammatic representation of an AED with a supplementary battery pack and an intermediate power control dongle in accordance with a second embodiment.

FIG. 5A is an electrical block diagram illustrating representative control electronics for the power control dongle of FIG. 4 in accordance with one embodiment.

FIG. 5B is an electrical block diagram illustrating representative control electronics for the power control dongle of FIG. 4 in accordance with another embodiment.

FIG. 6 is a flow chart illustrating a power delivery control protocol in accordance with another embodiment.

FIG. 7 is a diagrammatic representation of an AED with a supplementary battery pack in accordance with a second embodiment.

FIG. 8A is a perspective view of a portable AED carrying case designed to carry a supplemental power bank together with an AED. The carrying case is in the open position and holds the AED and power bank therein.

FIG. 8B is an exploded perspective view of the portable AED carrying case of FIG. 8A.

FIG. 9 is a block diagram illustrating electrical components of a representative defibrillator unit.

FIG. 10 is a block diagram illustrating electrical components of a representative interface unit.

In the drawings, like reference numerals are sometimes used to designate like structural elements. It should also be appreciated that the depictions in the figures are diagrammatic and not to scale.

### DETAILED DESCRIPTION

Automated External Defibrillators (AEDs) are typically designed with batteries that provide a shelf life of several years, often in the range of 2-4 years. These long-lasting batteries ensure that AEDs remain operational even after prolonged periods of inactivity, which is a critical requirement given the emergency-use nature of AEDs. However, this approach is primarily viable for traditional AEDs that do not offer extensive connectivity features and where batteries are designed to be replaceable, not rechargeable.

The Applicant has developed AEDs that provide a number of connectivity features, including periodic server check-ins, real-time status reporting, EMS integrations, etc. By way of example, U.S. Pat. No. 10,737,105 (P006A), U.S. Pat. No. 11,097,121 (P006B), U.S. Pat. No. 10,565,845 (P014A), U.S. Pat. No. 11,452,881 (P016A) and U.S. Pat. No. 11,839,770 (P016D1), each of which is incorporated herein by reference, describe a few such AEDs.

To help address the additional energy demands required for such connectivity and/or to help reduce their overall size, some AEDs utilize rechargeable batteries. This works very well when the AED is deployed at a location where it can be plugged into a charger or can otherwise readily be recharged. However, there are scenarios where an AED may be deployed at a location that does not have ready access to the power grid and/or recharging is less feasible.

To mitigate this challenge, the aforementioned U.S. Pat. No. 10,737,105(P006 A) and U.S. Pat. No. 11,097,121 (P006B) proposed integrating a supplemental battery pack (also referred to as a portable charger) into the AED system. Such a supplemental battery pack serves as an auxiliary energy source to recharge the AED's primary rechargeable battery. It also provides energy to recharge the batteries of an interface units incorporated into the AED system. Although such systems work well, there are continuing efforts to provide other supplemental power bank options that can further extend AED shelf life for a given size power bank and/or provide other desirable features. The present application describes power pack management techniques that can help extend the shelf life of rechargeable AEDs, as well as power packs, power pack accessories, and AED designs that implement such techniques.

### Supplemental Power Pack

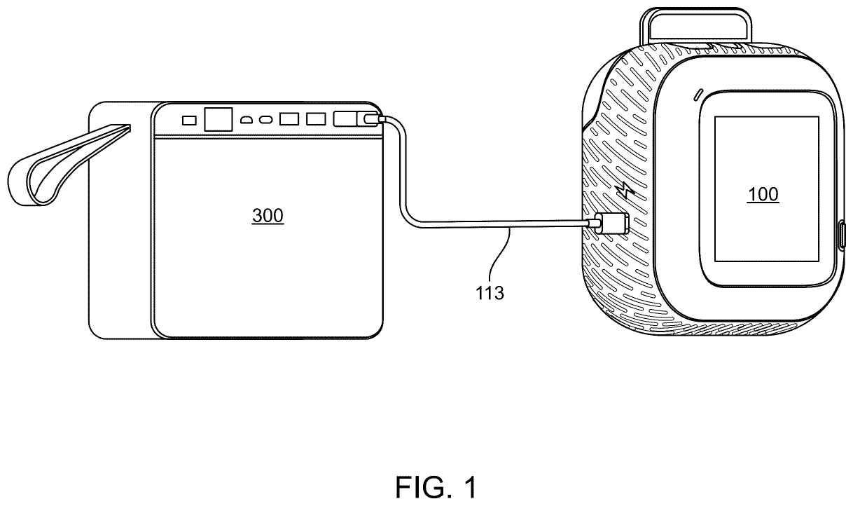

Referring next to FIG. 1, a supplemental power bank 300 for use in conjunction with an AED 100 having a rechargeable power supply will be described. In the illustrated embodiment, the supplemental power bank 300 is electrically connected to the AED system 100 via an electrical connector cable 113. Ideally, an “off the shelf” battery pack could be used as power bank 300. An inefficiency of using standard battery packs is that when the power bank 300 is turned on and connected to the AED, its power is always available. As such, there will be a small energy draw. Even though this “connection draw” may be small, over an extended storage period, measured in terms of weeks, months or years, the cumulative losses may be quite significant.

To help mitigate these losses, in the embodiment of FIG. 1, the power bank control electronics is designed to periodically activate and deactivate the output power supply in an automated manner. For example, in some embodiments, the power bank is deigned to periodically (e.g., once a day, once or twice a week, monthly, etc.) activate itself to make power available to the AED and thereafter deactivate itself (effectively turn itself off) after an appropriate interval. In some embodiments, the power bank deactivates itself after a designated interval (e.g., after it has been on for a designated period). The period of time that is appropriate for the power bank to remain active will vary based on factors such as the expected energy usage of the AED and the frequency of the power bank activations. In other embodiments, the power bank is configured to deactivate itself based on a sensed condition such as the charge current dropping below a designated level or that a predetermined charge level has been reached.

This periodic automatic activation and deactivation of the supplemental power bank's electrical connection with the AED is different than conventional battery packs which typically require user interaction to activate or deactivate the power supply. As will be appreciated by those familiar with the art, such manual intervention is unrealistic in the context of an AED deployed at a remote location.

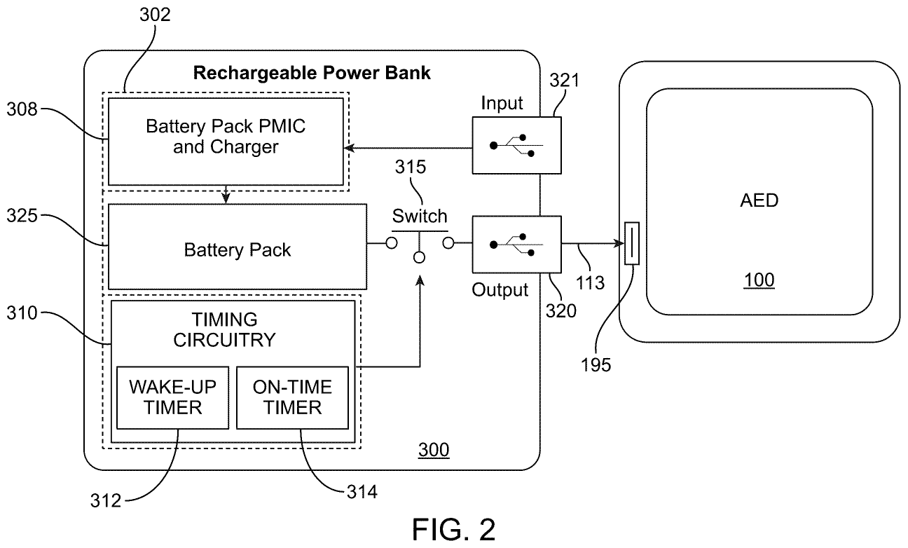

FIG. 2 illustrates selected electrical components of a representative power bank 300. In the illustrated embodiment, the power bank 300 is a rechargeable power bank and includes a power management controller/circuitry 302, an output port/connector 320, and power storage unit 325, which may take the form of a battery pack having one or more battery modules. In general, the power management controller/circuitry 302 is configured to automatically activate and deactivate the power bank as desired to make charging power available to the AED 100.

The power management controller/circuitry 302 can take a wide variety of different forms. In the embodiment illustrated in FIG. 2, the power management controller/circuitry 302 includes a power management integrated circuit and charger (PMIC) 308, timer circuitry 310, and an electrically activatable switch 315.

In the embodiment shown in FIG. 2, the PMIC 308 takes the form of a conventional power management integrated circuit and charger as commonly employed in battery packs and the timer circuitry 310 is added to provide the functionality described below. In other embodiments, the functionality of timer circuitry may be incorporated into the PMIC or a power management controller composed of one or more processors with associated memory.

In the illustrated embodiment, the switch 315 electrically couples the battery module 325 to output connector 320. When switch 315 is turned on, power from battery module 325 is available at connector 320, whereas when switch 315 is turned off, the connector is not powered thereby preventing the power bank from supplying power. The timer circuitry 310 is arranged to turn switch 315 on and off periodically on a defined schedule to thereby periodically activate and deactivate the power bank as will be described in more detail below.

In the illustrated embodiment, power bank connector 320 is a USB-C port and the connector cable 113 is a USB cable having USB-C connectors at its respective ends. In other embodiments, any of a variety of other types of connectors may be used (e.g., other USB connectors, Lightning connectors, MagSafe Connectors, mixed connectors, etc.), with the appropriate connectors/connector cables being dictated in large part by the nature of the power interfaces used by the AED and power bank respectively.

In the illustrated embodiment, the timer circuitry 310 includes a programmable wake-up timer 312 and a programmable “on-time” timer 314. In other embodiments the timers may be fixed, or a programmable wake-up timer and a programmable “on-time” timer may be incorporated into a processor that serves as the power management controller.

The wake-up timer 312 sets the frequency at which the power bank is activated. By way of example, in some preferred embodiments, the wake-up timer is set to activate the power bank on a designated interval. Wakeup intervals in the order of a range of hourly to monthly are believed to work well, although longer or shorter intervals may be appropriate in other circumstances. In one specific implementation, the wake-up timer turns on switch 315 once a day.

The “on-timer” 314 sets the period of time that the power bank remains on after it has been activated. By way of example, in some preferred embodiment, “on” times on the order of a minute to 100 minutes may be appropriate, although again, longer or shorter on-times may be used as appropriate and the appropriate length of the “on” time will vary based on how much charging is expected to be needed to maintain the AED battery(ies) at the desired charge state. In one specific example, the power bank is activated daily for a period of 2-5 minutes each day, as for example, 3 minutes.

In the illustrated embodiment, the power bank 300 itself is rechargeable and a separate charging input connector 321 is provided to facilitate recharging the power bank. When two separate connectors 320, 321 are provided, recharging can occur while the AED remains connected. In practice, in many embodiments, the connector 320 is actually a dual use connector that may be used for both supplying power to a connected device (e.g., the AED) and receiving power (e.g., for recharging the power bank battery). In some embodiments, multiple dual use connectors may be provided (e.g., connectors 320 and 321 may both receive power to recharge the battery pack 325 and supply power to external devices-such as the AED.)

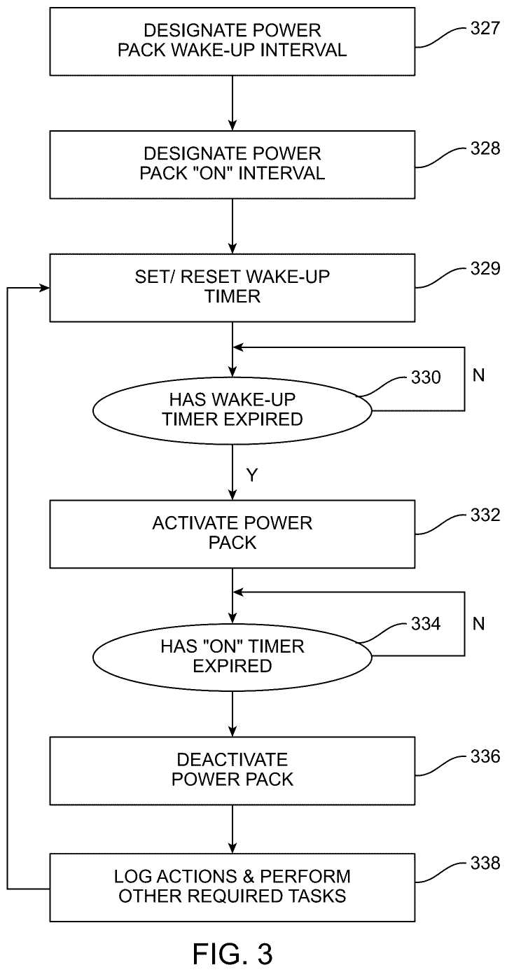

Referring next to FIG. 3, a representative power management protocol for maintaining the AED 100 in a reasonably charged state will be described. The interval setting of the wake-up and on-time timers is diagrammatically represented by blocks 327 and 328 respectively. In some embodiments, the “wake-up” and “on-time” timers are fixed or set programmatically at the power bank factory. In others, the times may be set programmatically by users, distributors or others. Such distributors may be an AED manufacturer, a distributor that supplies the power bank to a customer, etc.. At some point the wake-up timer is set and started as represented by block 329, thereby starting the process. This may be part of an initialization routine for the controller 302 when the power bank is turned on or initialized or at any other suitable time. For use, and AED is plugged into the power bank 300.

During operation, the system effectively waits for the wake-up timer to expire as represented by block 330 and then activates the power bank as represented by block 332. From a control standpoint, this can be accomplished in any suitable way. In some embodiments, separate timer circuity 310 including wake-up timer 312 and on timer 314 are provided. In other embodiments, a processor that functions as the controller 302 uses its own timer. In still other embodiments, the controller is placed in a sleep mode and a separate timer sends a wake-up interrupt to the controller when the timer counter hits the designated threshold (or counts down to zero or some other designated threshold), thereby prompting the controller to execute a suitable wake-up/activation protocol.

When the wake-up timer 312 triggers, the power bank is activated (“turned on”) as represented by block 332. This means that power from the battery module 325 is made available to the power output connector 320. In some embodiments, this is accomplished by the timer circuitry 310 (or alternatively the power bank controller) simply turning on switch 315 on to electrically connect battery module 325 to output connector 320 which presents power to input connector 195 on AED 100 via connector cable 113.

When the power bank is activated, the “on” timer 314 is started. The system effectively watches the “on-time” timer to determine when it expires as represented by block 334. When the timer expires (or hits a designated threshold), the power bank is effectively deactivated by turning off switch 315, thereby disconnecting the battery module from output connector 320 so that power is no longer available to the AED as represented by block 336. In some embodiments, the controller logs the actions taken and performs any other required tasks, as represented by block 338.

In the time period that the AED 100 has power, it charges its internal batteries in accordance with its existing charging protocols. By way of example, incorporated U.S. Pat. No. 10,737,105 (P006 A) and U.S. Pat. No. 11,097,121 (P006B) describe a few charging protocols that are appropriate for modular AED systems that incorporate a base defibrillator unit and an interface unit with separate rechargeable batteries.

Once the power bank has been “deactivated” (e.g., by turning off switch 315), the wake-up timer is reset (block 329) and the process is repeated. In some preferred implementations the PMIC 308 or battery pack controller is put to sleep between “active” or “on” periods to further minimize power consumption.

It should be appreciated that the timed power bank activation/deactivation control illustrated in FIG. 3 is representative in nature and can be implemented in a number of different ways as will be appreciated by those skilled in the art.

Since AEDs are typically expected to spend most of their lives in a standby state, their actual power consumption tends to be quite low. Therefore, they need very little charging to maintain a healthy charge state. The process illustrated in FIG. 3 substantially reduces the small losses associated with having a supplemental power bank persistently connected to the AED (referred to as the “connection losses”) because the power bank is on for a limited time at various intervals. This greatly reduces or eliminates, the “connection draw” that would otherwise sap energy from the power bank, thereby increasing the useful shelf-life of the power bank. Typically, the power bank would be active less 10% of the time, and more preferably, less than 5% of the time. In, some preferred implementations, the power bank is active less than 1% of the time. By way of example, on-time percentages on the order of 0.1% to 1% of the time are suitable for many applications. This substantially reduces the power leakage that naturally occurs when the connection is powered. Our experiments have shown that for some specific power banks, the useful charging life of the power bank can be extended dramatically using the described approach.

Another advantage of the described power management approach is that it does not require any modifications to the AED 100 itself which is desirable because any modifications to an AED will typically require regulatory approval (such as FDA approval in the United States), which can present a barrier to adoption.

### Dongle-based Power Management

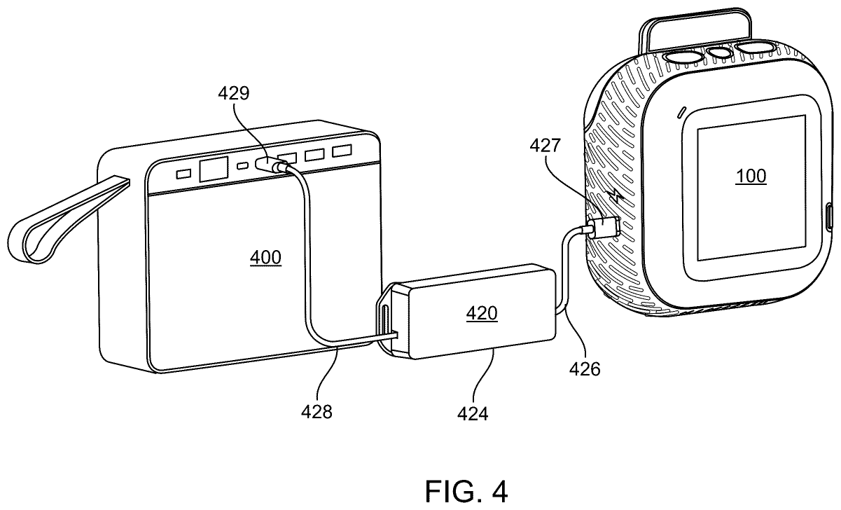

A potential drawback of the power management approach described in FIG. 3 is that it requires some customization of the power bank in order to provide the described functionality. This means that normal, off-the-shelf battery-based power banks cannot be used for this purpose, which potentially increases the costs of the power bank and limits the available power banks that can be used. Referring next to FIG. 4, an alternative dongle-based embodiment will be described that does not require modification or customization of either the AED or the off-the-shelf power banks. In this embodiment, a dongle 420 is used to provide some power management functions.

In the illustrated embodiment, the dongle 420 includes a main body 424 and a pair of connector cables 426 and 428 that plug into/connect with the AED 100 and supplemental power bank 400 respectively. In the illustrated embodiment, the connector cables 426 and 428 are connected to the main body and have USB-C connectors 427, 429 at their respective free ends. In other embodiments, other types of power interfaces may be used (e.g., other USB connectors, Lightening connectors, MagSafe Connectors, wireless power interfaces, etc.), or one or both of the connector cables 426 and 428 may be replaced by simple connectors or wireless power interfaces.

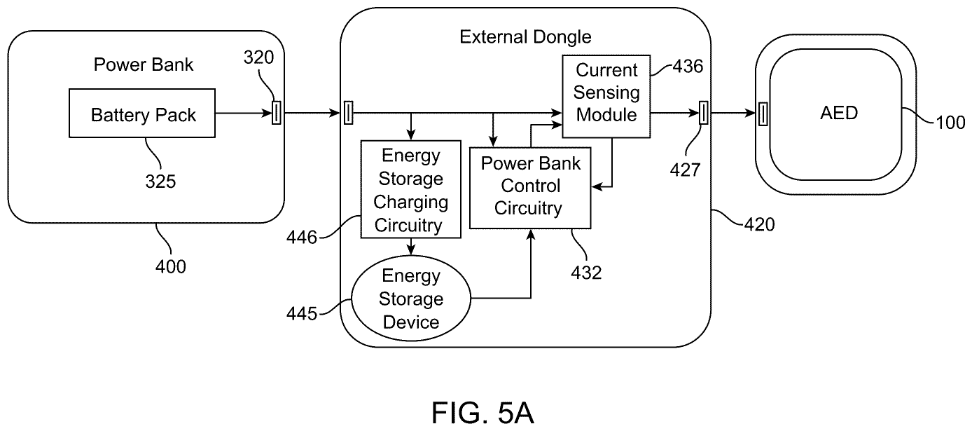

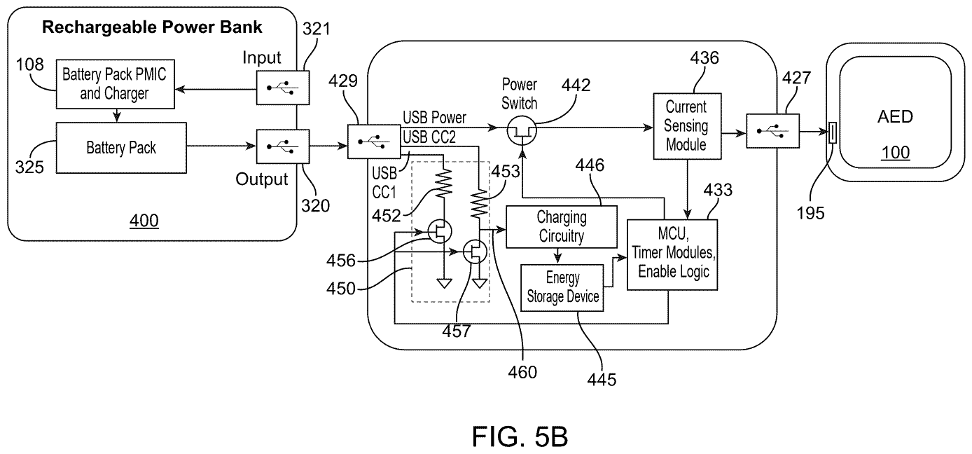

FIG. 5A illustrates a dongle-based power bank management architecture. In the illustrated embodiment, dongle 420 includes a dongle control unit 432, a current sensing module 436, a power switch 442, a dongle energy storage device 445, battery charger circuity 446. The dongle control unit 433 includes the timer and control logic that controls the supply of power to the AED 100, connection sensors (as required) and power bank enable/disable circuitry or functionality as described in more detail below. In some embodiments, the dongle control unit takes the form of or include a Microcontroller Unit (MCU) or other processor together with one or more timer modules and enabling logic. In other embodiments, a programmed processor, an application specific integrated circuit (ASIC) or other control circuitry may be provided to perform the described functions.

In general, the dongle 420 is configured to manage the recharging of the AED 100. As such, somewhat analogously to the controller 302 of FIG. 3, the dongle control unit 432 periodically wakes up and activates the power bank 400 to thereby cause power to be delivered from the power bank 400 to the AED 100 to facilitate recharging the AED. The dongle control unit subsequently deactivates the power bank 400 at an appropriate time to help prolong the power bank's useful charging life.

As will be appreciated by those familiar with power packs, many conventional power banks are configured to deactivate themselves (turn off their supply of power to output connector 320) when no connected devices are detected. Therefore, in some embodiments, the dongle control unit includes power bank enable/disable circuitry or functionality that is used to trigger the power bank to activate and deactivate itself on command. When the enable/disable functionality is activated, conventional circuitry on the power bank senses that a device is connected to its output connector 320 and therefore activates itself. When the enable/disable functionality is deactivated, the power bank is fooled into thinking that nothing is connected to its output connector 320 and therefore deactivates itself. One representative embodiment of enable/disable circuitry is described below with respect to FIG. 5B. However, it should be appreciated that in other embodiments the enable/disable functionality can be implemented using a variety of other specific circuitry or control logic. In some embodiments, the enable/disable functionality is performed algorithmically by a processor that serves as dongle control unit 432.

In some embodiments, the power bank is activated and deactivated on a predetermined schedule as discussed above. However, in other embodiments, the duration of the charging may be based on the needs of the receiving AED rather than simply being a preset time. That is, the power pack is deactivated when the AED is deemed sufficiently charged instead of at a fixed time. In some embodiments, the current sensing module 436 is used to help determine when the AED is sufficiently charged. In some implementations, recharging is deemed sufficiently complete when the charging current drawn by the AED falls below a designated threshold. In the illustrated embodiment, the current sensing module 436 senses the current being delivered to the AED at any given time and reports that information to the dongle control unit 432. Thus, when the charging current falls below the threshold, the dongle control unit 432 directs the power pack to deactivate itself thereby concluding a charging cycle. The current sensor may take any desired form. By way of example, shunt resistor type current sensors, hall effect sensors and others may be used in different embodiments.

As previously discussed, charging cycles may be initiated at regular intervals. Additionally, when desired, charging cycles may be initiated at other appropriate times, as for example, when a new AED or power bank connection is detected. That is, when the dongle is newly plugged into an AED or power bank such that both the AED and power bank are connected.

In some embodiments, connection sensors are provided to detect when the connector cables 426/428 are plugged into/connected with an associated device (AED 100 and supplemental power bank 400 respectively). When the connector cables are USB-C connector cables, standard CC pin control and processing may be used to detect both the connection of a device to one of the dongle's connector cables and the orientation of such connection. Other suitable connection sensors may be used as appropriate when other types of connectors are used.

The dongle energy storage device 445 provides power to the electrical components of the dongle, including, most notably, the dongle control unit 432. The power requirements of the dongle are very low, so the dongle energy storage device may be very small. The dongle energy storage device may take any suitable form. For example, coin cell type batteries, supercapacitors, etc. work well. In some embodiments, the dongle energy storage device 445 is rechargeable and charging circuitry 446 is provided to maintain the dongle battery in a suitably charged state using power received from the power bank 400. This is helpful because, like the AED 100 that the dongle is connected to, there is an expectation that the dongle 420 will remain operation over an extended shelf life measured in terms of multiple years.

Referring next to FIG. 5B another suitable dongle architecture will be described. In the illustrated embodiment, dongle 420 includes input connector 427, output connector 429, a control unit 433, a current sensing module 436, a power switch 442, a dongle energy storage device 445, battery charger circuity 446, connection sensors (not shown), a power bank enable/disable circuitry 450 (sometimes referred to as a disconnect spoofing circuit) and a parasitic energy storage device charging circuit 460.

The control unit 433 includes the timer and control logic that controls the activation and deactivation of the power bank, and thus the supply of power to the AED 100, as well as other dongle functionality.

The power switch 442 is optional and is controlled by control unit 433. When desired, the control unit can turn off power switch 442 off to cut the supply of power to the AED 100. The current sensing module 436 functions as described above with respect to FIG. 5A. The power bank enable/disable circuitry 450 acts as a spoofing circuit that can (upon command) fool the power bank into thinking that nothing is connected thereto, which prompts the power bank to deactivate itself.

In the illustrated embodiment, the connectors 427 and 429 take the form of USB-C connectors, although as previously indicated, other connectors may be used in other embodiments. The USB-C protocol defines a configuration channel that includes two configuration channel pins CC1 and CC2. These pins are used to communicate and negotiate features like power delivery, data roles and connection orientation. In general, an open circuit indicates that no cable or device is connected. For devices that receive power, a specific resistance indicates that a device is a sink device (power consumer). For devices that provide power, specific resistances indicate different maximum currents that may be supplied by the device. The dongle 420 acts as a sink device with respect to supplemental power bank 400 and as a source device with respect to AED 100.

In the illustrated embodiment, pull-down resistors 452 and 453 are connected to the CC1 and CC2 lines of input connector 429 respectively to thereby designate the dongle 420 as a power consumer relative to the supplemental power bank 400. The disconnect spoofing circuit 450 incorporates resistors 452 and 453, as well as switches 456 and 457 respectively, which are positioned electrically between their corresponding resistors and ground.

If/when the dongle control unit 433 determines that power is not needed, it turns off switches 456 and 457. When switches 456 and 457 are turned off, the CC1 and CC2 lines of input connector 429 present the appearance of an open circuit (or near open circuit) to the supplemental power bank's output connector 320. This fools the power pack controller (PMIC 408) into thinking that nothing is connected to output connector 320. As previously stated, many conventional power banks are configured to deactivate themselves (turn off their supply of power to output connector 320) when no connected devices are detected. Therefore, when switches 456 and 457 are turned off, the battery pack controller (PMIC 408) turns off the supply of power to connector 320 because it perceives that nothing is connected thereto. This eliminates the vast majority of the connection losses associated with having the dongle or an AED “plugged-in” to the power bank.

If/when the dongle control unit 433 determines that power is desired, it turns on switches 456 and 457, which causes the standard defined current to flow through CC1 and CC2. That current flow causes the power bank to sense that a device is now connected to output connector 320. In response thereto, the power bank activates (turns on), thereby providing power to the output connector 320 and the dongle, which is connected thereto. Many conventional power banks are designed to activate in this manner when a new connection is detected and the spoofing circuit 450 triggers this existing power bank functionality by selectively turning switches 456 and 457 on and off as appropriate.

In the illustrated embodiment, a resistor/switch pair is provided on each CC line. However, in other embodiments, a single resistor/switch pair (e.g., resistor 452/switch 456 or resistor 453/switch 457) may be provided instead of both resistor/switch pairs. Although a particular embodiment is shown, it should be appreciated that a variety of alternative circuit structures can be used to provide the desired spoofing.

Another unique feature of the dongle 420 is the parasitic battery charging circuit 460. Specifically, the dongle's energy storage device charger circuitry 446 uses one (or both) of the configuration control lines as its “power supply” for charging/recharging the dongle energy storage device 445. In the illustrated embodiment, the energy storage device charger 446 is connected to CC2 downstream of resistor 453. In practice, many conventional power banks maintain the configuration control lines (CC1 and CC2) active when the power bank is in a sleep mode with its output power turned off to facilitate detecting if/when devices are connected thereto. Since, CC2 remain “live” in this sleep mode, a very small current will be parasitically drawn by the dongle's energy storage device charger circuitry to thereby facilitate trickle charging the dongle energy storage device 445. Thus, the dongle's energy storage device 445 is charged parasitically while the power bank is in its sleep mode. In practice, the amount of power consumed by the dongle is very low and this parasitic charging off of one of the configuration control lines is sufficient to maintain the dongle battery's charge level. However, if more charging is desired, both CC lines may be used as supplies for the battery charger circuitry 446 and/or power from the USB power line can be used to supply supplemental dongle energy storage device charging power.

It should be appreciated that the parasitic charging of the dongle energy storage device can readily be configured to charge all the time, only when the power bank is in the sleep mode, only when the power bank is active, or at any other desired times/time intervals.

Although the embodiment shown in FIG. 5B has one connector configured as a sink for coupling to the supplemental power bank, and the other configured as a source for coupling to the AED, it should be appreciated that in other embodiments, the connectors may be configured for dual roles such they can each act as either a source or sink. In such cases, the described disconnect spoofing circuit 450 and parasitic energy storage device charging circuit 460 may be included in the sink electronics associated with each connector 427, 429.

In some embodiments, 433 the control unit takes the form of a Microcontroller Unit (MCU) or other processor together with one or more timer modules and enabling logic. In other embodiments, a programmed processor, an application specific integrated circuit (ASIC) or other control circuitry may be provided to perform the described functions.

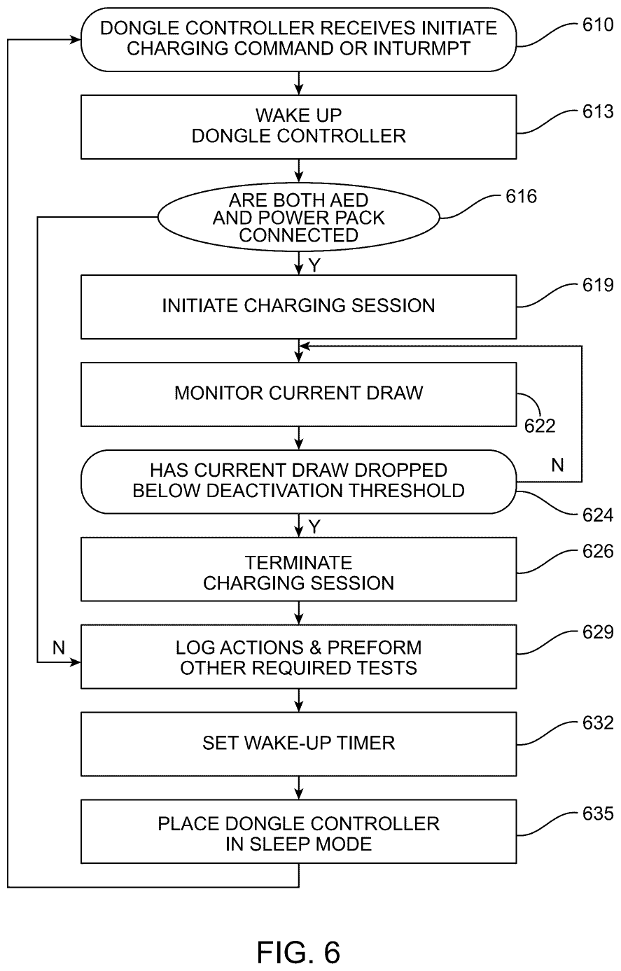

Referring next to FIG. 6, a representative charge control protocol 600 suitable for implementation by the dongle 420 will be described. The process begins when a processor (or other suitable component in dongle control unit 433) receives an initiate charging protocol command or interrupt (block 610). In some circumstances, the initiate charging protocol command takes the form of an interrupt received from a sleep timer that indicates that a designated sleep period has expired. In other circumstances, the initiate charging protocol command may take the form of an interrupt from a connection sensor indicating that a new connection has been detected with either an AED 100 or power bank 400. Of course, in other embodiments, the timer may be implemented by the processor itself or the initiate charging protocol command/interrupt can come from other appropriate sources.

When the initiate charging protocol command/interrupt is received, the processor is awaken from its sleep mode if it is not already awake. Block 613. The processor then checks to verify that both an AED and a power bank are connected to the dongle. Block 616. When both a power bank and an AED are connected (the yes branch from block 613), the processor initiates a charging session. Block 619. In embodiments in which the power bank 400 is always or already on, this is accomplished by turning on the power connection switch 442 which makes an electrical power connection between the power bank 400 and AED 100. In embodiments where the power bank is in a sleep or deactivated mode, the power bank is woken up so that it begins supplying power. When the spoofing circuit 450 of FIG. 5B is used, the this is accomplished by turning on spoofing switches 456, 457. In other embodiments, the power bank may be activated in other ways. Typically, the power bank will be activated before the power connection switch 442 is turned on, although that is not a requirement.

When power is available from the power bank, the AED detects the presence of power on its connector port 195 and initiates its battery charging protocol. As previously described, the specific charging protocols employed by the AED's may vary widely based on the design and needs of the AED, but from the perspective of dongle 420, the dongle serves its purpose of making recharging power available to the AED for use as the AED sees fit.

After the switch 442 has been turned on, the processor monitors the AED's current draw via current sensing module 436. Block 622. The draw current may be measured using any of a variety of conventional current sensors. If/when the current draw drops to or below a predefined charge termination threshold (decision block 624), switch 442 is turned off (block 626) thereby terminating the charging session. The charge termination threshold is selected based on the expected charging needs and characteristics of the AED's battery(ies). By way of example, in one particular embodiment, an AED having two rechargeable batteries (e.g., one in a base defibrillator unit and a second in an attached interface unit) each of which are configured to draw up to 1 amp for charging purposes, an appropriate charge termination threshold may be on the order of 0.4 amps. Although a specific example is given, it should be appreciated that the actual charge termination threshold may vary widely based on the nature of the batteries used in the AED, as well as the desired charging protocols and charge levels.

In embodiments where the power bank deactivated after the charging session terminates, appropriate steps are taken to initiate the power bank's deactivation. When the spoofing circuit 450 of FIG. 5B is used, the this is accomplished by turning off spoofing switches 456, 457. In other embodiments, the power bank may be deactivated in other appropriate ways.

During the charging session and after the session terminates, the processor 433 preferably logs significant events relative to the session and performs any other desired tasks as represented by block 629. The logged events may include timestamps indicating when the charging session starts and finishes, current draws, measured voltages of the power bank (which may be useful in determining the charge status of the power bank), etc.

The processor wake-up timer is reset (block 632) and the processor is transitioned to its sleep mode (block 635). The process then repeats when the wake-up timer expires.

It is noted that for ease of explanation, the logging, wake-up timer resetting, and other administrative steps are shown as occurring after the charge session terminates. However, it is appreciated that in many embodiments, many of these tasks will actually be performed during the charging session and some may be performed before charging actually begins or during charging.

In the embodiment described with reference to FIG. 3, the power bank 300 is configured to periodically wake up and activate itself (i.e., make power available to connected device) for a designated period of time and then deactivate itself and go back to sleep until it is next awakened. In the embodiment described with reference to FIG. 6, the dongle 420 periodically wakes up in a similar manner, but deactivates when the current falls to or below a designated threshold. It should be appreciated that in other embodiments, the logic for activating and/or deactivating the availability of power for charging can be incorporated into any of the supplemental battery pack, the power delivery accessory (e.g., dongle 420) or the AED itself. In any of these embodiments, the deactivation can be triggered based on elapsed time, sensed current draws dropping below a designated threshold, or in any other suitable manner. Thus, the time base power bank activation/deactivation mechanism may be incorporated into any of the power bank itself, the power delivery accessory or the AED. Similarly, the current draw-based power shut-off can be incorporated into any of the power bank, the power delivery accessory, or the AED.

### AED Based Power Management

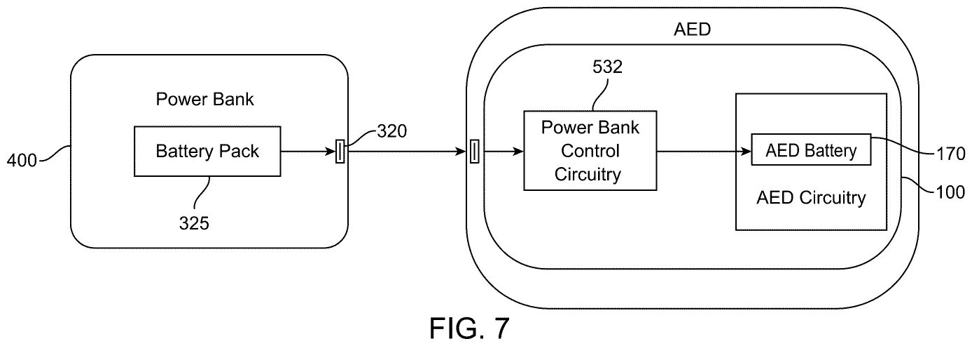

The power pack and dongle-based power pack power management techniques described above both work well, however they generally require either customization of the power bank or the provision of an additional component-e.g., the dongle. In other embodiments, the described power management functionalities can be incorporated into the AED itself. Such an arrangement is diagrammatically illustrated in FIG. 7.

In the system of FIG. 7, a conventional power bank 400 may be used to supply supplemental charging power to the AED 100. This embodiment differs from the previously described embodiments in that the power bank control circuitry 532 is integrated into the AED. The power bank control circuitry 532 may be substantially the same as the power bank control circuitry 432 discussed above with respect to the dongle embodiment with the primary difference being that it is incorporated into the AED itself, rather than being housed in a separate component (e.g., the dongle).

### Carry Case With Integrated Power Bank

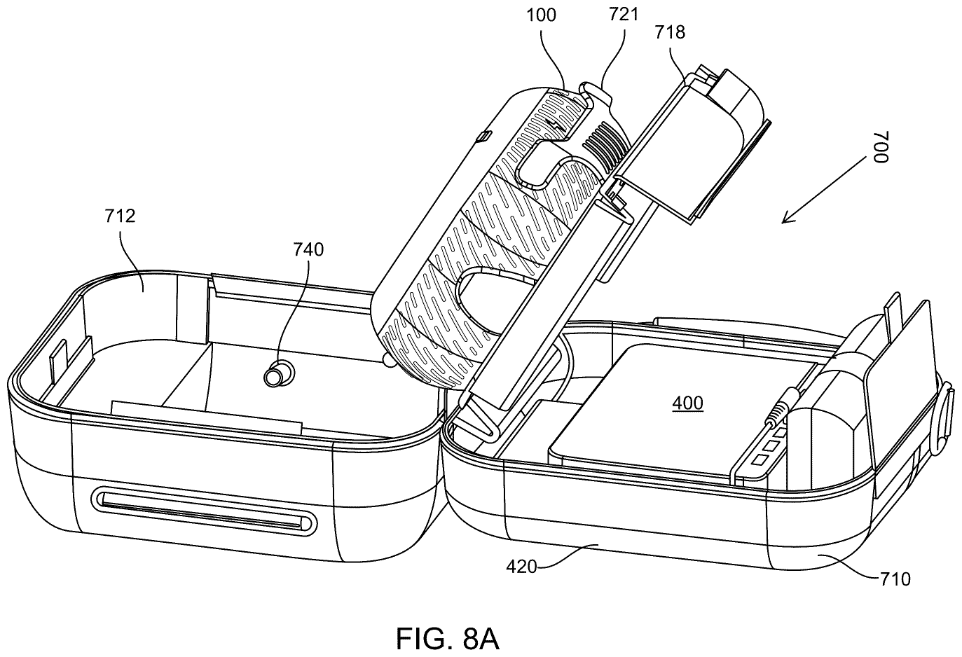

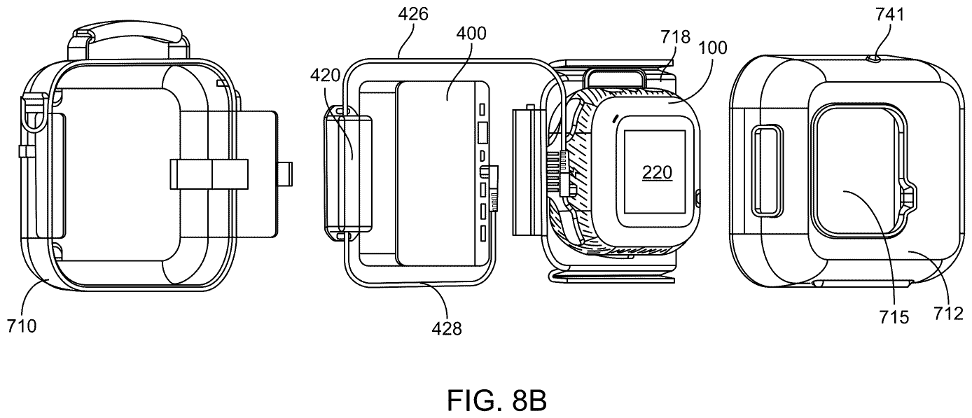

FIGS. 8A and 8B are perspective and exploded perspective views respectively of an AED carrying case 700 that is designed to carry a supplemental power bank together with the AED. The carrying case 700 includes a base 710, a cover 712 having a window 715 therein, and an AED support insert 718 having a bracket 721 that receives the AED. The bracket 721 is sized so that it firmly grips the AED when the AED is placed therein. Preferably the bracket 721 has resilient bracket arms that allow the AED to be press-fit into the bracket. The window 715 and bracket 721 are positioned relative to one another such that when AED 100 is placed in the bracket, a display screen 220 of the AED is exposed through window 715 such that the display screen can be viewed even when the AED is stored within the closed case. In some embodiments, the window 715 is open so that the display screen (which may be a touch sensitive display screen) can also be accessed through the window.

The supplemental power bank 400 and any dongle 420 are stowed in the base 710 behind the AED support insert 718. In some embodiments, the AED support insert 718 is sized such that it rests on the supplemental power bank 400 in the stowed position to thereby provide additional mechanical support for the AED.

The AED 100 includes an indicator light that is used to convey its operational status. For example, the indicator light may be green, or periodically flash green when the AED is in good working order, but may be red or flash red when the AED is not operational. Of course, other colors and/or flashing patterns may be used to convey other operation states. When the AED is in its carrying case 700, it can be difficult to see the indictor light. To improve visibility of the indicator light, a light pipe 740 is provided on the carrying case at a position adjacent where the indicator light is located when the AED is stowed in the carrying case. More specifically, the input (coupling) end of the light pipe is position adjacent the indicator light. The output end includes a 741 lens or diffuser that is designed to disperse the light and make it visible to from all angles. In some embodiments, diffuser lens 741 is domed-shaped lens 741.

### Automated External Defibrillator

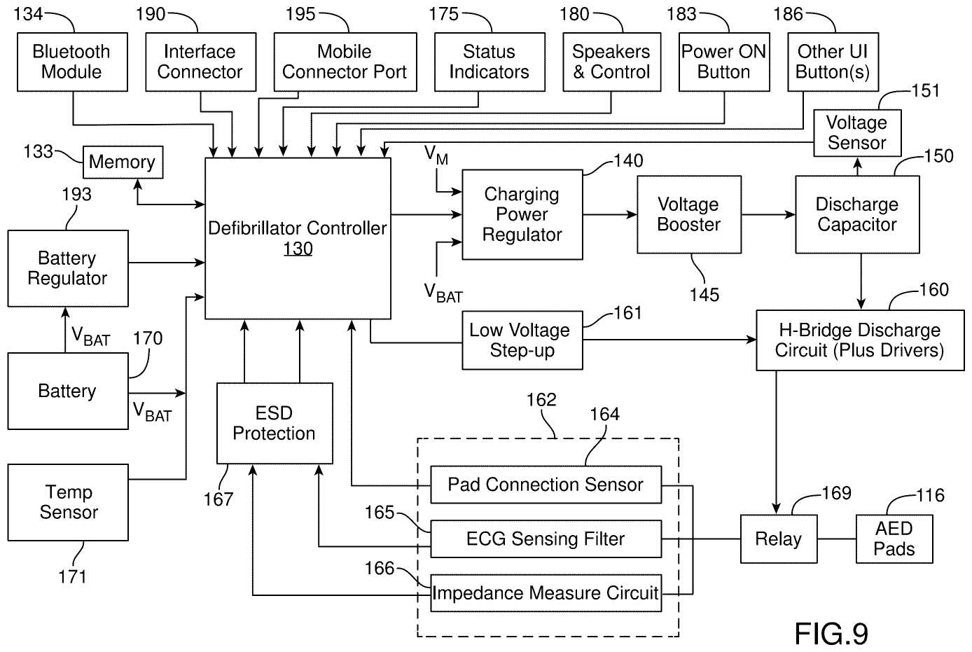

In some embodiments, the AED 100 includes a base defibrillator unit 110 having an interface unit 200 secured thereon. FIG. 9 is a block diagram illustrating one representative electronics control architecture and associated components suitable for use in the base defibrillator unit 110. In the illustrated embodiment, the electronic components include a defibrillator controller 130, memory 133, a wireless communications module in the form of Bluetooth module 134, a charging power regulator 140, a voltage booster 145 (which may have multiple stages), a high voltage capacitor 150 for temporarily storing sufficient electrical energy suitable to provide a defibrillation shock, discharge control circuitry 1160, pad related sensing circuitry 162 and relays 169, power storage unit 170, battery regulator 193, status indicator(s) 175, speaker(s) 180 and one or more electrical connectors (e.g., interface connector 190, mobile connector port 195, charger connector (not shown), etc.). The charging power regulator 140 and voltage booster 145 which cooperate to control the charging of the shock discharge capacitor 150 are sometimes referred to herein as a charging circuit.

The defibrillator controller 130 is configured to control the operation of the base defibrillator unit and to direct communications with external devices, as appropriate. In some embodiments, the defibrillator controller includes one or more processors arranged to execute software (some or all of which may take the form of firmware) having programmed instructions for controlling the operation of the base unit, directing interactions with a user and communications with external components. The software may be installed on the memory 133. Although the singular term memory is often used herein, it should be appreciated that the memory may be divided into multiple different parts which take any suitable form or combination of forms (e.g., various types of RAM, ROM, PROM, EEPROM, etc.) Unless the context suggests otherwise, references to “memory” herein are intended to cover all suitable forms and combinations of physical memory. Similarly, although the singular term “processor” is often used herein, it should be appreciated that any appropriate number of processors and/or processing cores can be utilized and unless the context suggests otherwise, references to “processor” herein are intended to cover processing units composed of one or more physical processors or processing cores.

The base defibrillator unit 110 may optionally be configured so that it is capable of drawing power from certain other available power sources beyond power storage unit 170 to expedite the charging of shock discharge capacitor 150. The charging power regulator 140 is configured to manage the current draws that supply the voltage booster, regardless of where that power may originate from. For example, in some embodiments, supplemental power may be supplied from a mobile device coupled to mobile connector port 195 or from a portable charger/supplemental battery pack coupled to charger connector 197.

The voltage booster 145 is arranged to boost the voltage from the operational voltage of power storage unit 170 to the desired operational voltage of the discharge capacitor 150, which in the described embodiment may be on the order of approximately 1400V-2000V (although the defibrillator may be designed to attain any desired voltage). In some embodiments, the boost is accomplished in a single stage, whereas in other embodiments, a multistage boost converter is used. A few representative boost converters are described in the incorporated U.S. Pat. No. 10,029,109. By way of example, in some embodiments, a flyback converter, as for example, a valley switching flyback converter may be used as the voltage booster 145—although it should be appreciated that in other embodiments, a wide variety of other types of voltage boosters can be used.

A voltage sensor 151 is provided to read the voltage of the capacitor 150. The voltage sensor 151 may take the form of a voltage divider or any other suitable form. This capacitor voltage reading is utilized to determine when the shock discharge capacitor 150 is charged suitably for use. The sensed voltage is provided to controller 130 which determines when the capacitor 150 is charged sufficiently to deliver a defibrillation shock. The capacitor 150 can be charged to any desired level. This can be useful because different defibrillation protocols advise different voltage and/or energy level shocks for different conditions. Furthermore, if the initial shock is not sufficient to restart a normal cardiac rhythm, some recommended treatment protocols call for the use of progressively higher energy impulses in subsequently administered shocks (up to a point).

The discharge circuitry 160 may take a wide variety of different forms. In some embodiments, the discharge circuitry 160 includes an H-bridge along with the drivers that drive the H-bridge switches. The drivers are directed by defibrillator controller 130. The H-bridge outputs a biphasic (or other multi-phasic) shock to patient electrode pads 116 through relays 169. The relays 169 are configured to switch between an ECG detection mode in which the patient electrode pads 116 are coupled to the pad related sensing circuitry 162, and a shock delivery mode in which the patient electrode pads 116 are connected to H-Bridge to facilitate delivery of a defibrillation shock to the patient. Although specific components are described, it should be appreciated that their respective functionalities may be provided by a variety of other circuits.

The pad related sensing circuitry 162 may include a variety of different functions. By way of example, this may optionally include a pad connection sensor 164, ECG sensing/filtering circuitry 165 and impedance measurement chip/block 166. The pad connection sensor is arranged to detect the pads are actually connected to (plugged into) the base defibrillator unit 110. The ECG sensing/filtering circuitry 165 senses electrical activity of the patient's heart when the pads are attached to a patient. The filtered signal is then passed to defibrillator controller 130 for analysis to determine whether the detected cardiac rhythm indicates a condition that is a candidate to be treated by the administration of an electrical shock (i.e., whether the rhythm is a shockable rhythm) and the nature of the recommended shock. When a shockable rhythm is detected, the controller 130 directs the user appropriately and controls the shock delivery by directing the H-bridge drivers appropriately.

In some embodiments, the power storage unit 170 takes the form of one or more batteries such as rechargeable Lithium based batteries including Lithium-ion and other Lithium based chemistries, although other power storage devices such as one or more supercapacitors, ultracapacitors, etc. and/or other battery chemistries and/or combinations thereof may be used as deemed appropriate for any particular application. The power storage unit 170 is preferably rechargeable and may be recharged via any of a variety of charging mechanisms. In some embodiments, the power storage unit 170 takes the form of a rechargeable battery. For convenience and simplicity, in much of the description below, we refer to the power storage unit 170 as a rechargeable battery. However, it should be appreciated that other types of power storage devices can readily be substituted for the battery. Also, the singular term “battery” is often used, and it should be appreciated that the battery may be a unit composed of a single battery or a plurality of individual batteries and/or may comprise one or more other power storage components and/or combinations of different power storage units.

In some embodiments, the base defibrillator unit 110 is capable of drawing power from other available power sources for the purpose of one or both of (a) expediting the charging of shock discharge capacitor 150 and (b) recharging the power storage unit 170. In some embodiments, the battery can be recharged using one or more of the externally accessible connector ports 195, a dedicated charging station, a supplemental battery pack (portable charger), an interface unit 200, etc. as will be described in more detail below. When wireless charging is supported, the base defibrillator unit may include a wireless charging module 174 configured to facilitate inductive charging of the power storage unit 170 (e.g., using an inductive charging station 294, or other devices that support inductive charging, as for example an inductively charging battery pack, a cell phone with inductive charging capabilities, etc.).

The base unit also includes a number of software or firmware control algorithms installed in memory 133 and executable on the defibrillator controller. The control algorithms have programmed instructions suitable for controlling operation of the base unit and for coordinating the described broadcasts, as well as any point-to-point communications between the base unit 110 and the interface unit 200, connected devices, and/or any other attached or connected (wirelessly or wired) devices. These control routines include (but are not limited to): communication control algorithms, heart rhythm classification algorithms suitable for identifying shockable rhythms; capacitor charge management algorithms for managing the charging of the discharge capacitor; capacitor discharge management algorithms for managing the delivery of a shock as necessary; user interface management algorithms for managing the user instructions given by the defibrillator and/or any connected user interface devices (e.g. interface unit 200, mobile communication device 105) during an emergency; battery charge control algorithms for managing the charging of power storage unit 170; testing and reporting algorithms for managing and reporting self-testing of the base unit; software update control algorithms and verification files that facilitate software updates and the verification of the same.

In many installations, an AED will be expected to be stored for long periods of time without being plugged into power and therefore relying solely on battery power. Accordingly, it is important to minimize the power drain as much as possible during this time. In some embodiments, the defibrillator controller is configured to shut down power to all electrical components (including itself) except for (a) a real time clock (not shown), (b) the Bluetooth module 134, and (c) a power controller (not shown) when the AED is in the standby (resting) mode. In some embodiments, the clock and/or the power controller are integrated into the Bluetooth module 134 or an I/O adaptor board that includes the Bluetooth module. The clock is configured to periodically send a status check alarm to the power controller which wakes the power controller up from a sleep mode when the power controller is separate from the Bluetooth module. In response to the status check alarm, the power controller restores power to the entire system. Once power is restored, the defibrillator controller 130 performs a status check and instructs the Bluetooth module 134 to update the standby status message 150 as appropriate. After the status check is performed, the defibrillator controller will again shut down power to all electrical components except the clock, the Bluetooth module 134 and the power controller. The status check alarms can be generated at any desired intervals, as for example, once each day.

With the described power management scheme, the Bluetooth module 134 can continue to broadcast standby status messages even when the AED is powered down.

If the Bluetooth Module 134 makes a connection while the AED is in the standby mode, it sends a message to the power controller, which in turn, powers on the system, thereby allowing the defibrillator controller or other suitable component to communicate with the connecting device. Thus, the AED can effectively be woken up via a Bluetooth connection request. Of course, the power controller is also configured to power the system when a user presses the AED's “ON” button or otherwise activates the AED.

In other embodiments, one or more of the electrical components of the AED, such as the defibrillator controller 130, can be placed in a “sleep” mode rather than being turned off when the AED is in the standby mode. However, a significant advantage of actually turning the defibrillator controller and the bulk of the AED's electrical components off as opposed to placing them in a sleep mode is that it eliminates the power draw associated with the sleep mode, thereby potentially extending the AED's shelf life. It should be appreciated that the power controller/power down approach can be also used with AEDs that don't incorporate a Bluetooth module and/or support the low energy broadcasts described herein.

In some embodiments, a temperature sensor 171 is provided within the defibrillator itself for detecting the internal temperatures of the AED (as opposed to an environmental temperature), which is then used in the temperatures used to trigger the temperature fault notification and clearance messages. Preferrable the temperature sensor is positioned adjacent one of the more temperature sensitive components such as battery 170 so that the reported temperature is directly related to the internal temperature of the AED near the temperature sensitive component.

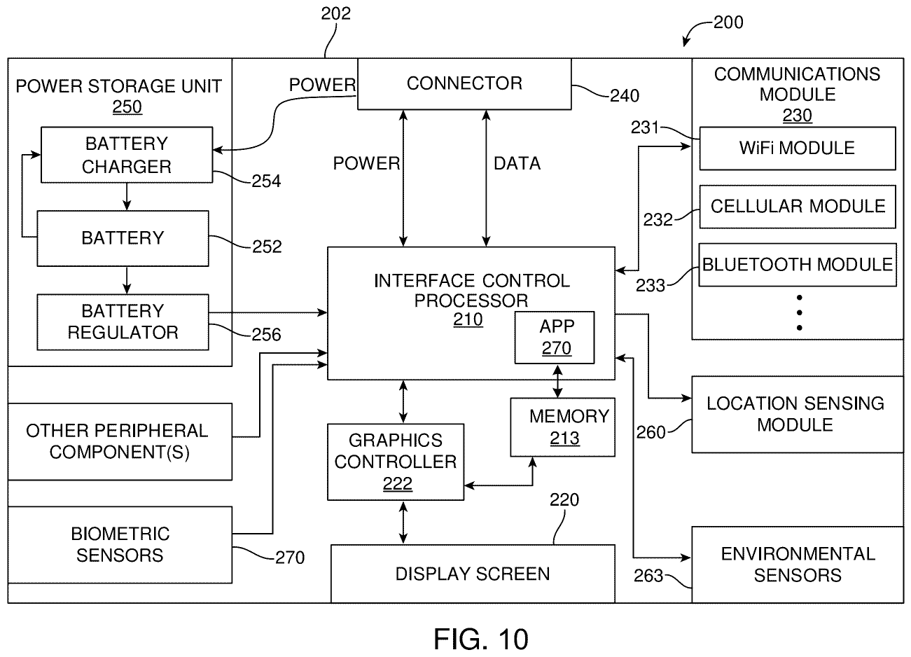

FIG. 10 illustrates some of the electrical components of a representative interface unit 200. In the illustrated embodiment, the interface unit 200 includes an interface controller (processor) 210, memory 213, a display screen 220, a communications module 230, an electrical connector 240, an interface unit power storage unit 250, and a location sensing module 260, all of which may be housed within the interface unit housing 202. The interface unit has software (which may take the form of firmware and/or an app 270) installed or installable in memory 213 to provide programmed instructions suitable for controlling operation of the interface unit and for coordinating communications between the interface unit 200 and the base defibrillation unit 110 and/or remote devices to provide the described functionalities of the interface unit.

The processor 210 controls operation of the interface unit and coordinates communications with both the base unit 110 and remote devices such as a central server (as will be described in more detail below). In some embodiments, the processor 210 is arranged to execute a defibrillator app 270 or other software that can be used both during use of the defibrillator system 100 during a cardiac arrest incident and to facilitate non-emergency monitoring or/or use of the defibrillator system 100. Similar to the base unit processor 130 discussed above, unless the context suggests otherwise, the processor 210 may take the form of a single processor, multiple processors, multiple processing cores and other processing unit configurations.

The display screen 220 is a touch sensitive screen suitable for displaying text, graphics and/or video under the direction of the processor 210 to assist both during both emergency situations and at other times. The touch sensitive screen is configured to receive inputs based on a graphical user interface displayed thereon. In some embodiments an optional graphics controller 222 may be provided to facilitate communications between the interface control processor 210 and the display screen 220. In other embodiments, the functionalities of the graphics controller may be part of the processor 210.

The communication module 230 is provided to facilitate communications with remotely located devices such as the central server. The communications module 230 may be configured to utilize any suitable communications technology or combination of communication technologies including one or more of cellular communications, Wi-Fi, satellite communications, Bluetooth, NFC (Near Field Communications), Zigbee communications, DSRC (Dedicated Short-Range Communications) or any other now existing or later developed communications channels using any suitable communication protocol. By way of example, in the illustrated embodiment, the communications module 230 includes Wi-Fi, cellular and Bluetooth modules 231, 232 and 232 that facilitate Wi-Fi, cellular and Bluetooth communications respectively.

The electrical connector 240 is configured to mate with interface connector 190 on the base defibrillator unit 110. The connectors 190 and 240 are configured to facilitate communications between the defibrillator controller 130 and the interface unit's processor 210. The connectors 190 and 240 are also preferably arranged to supply power from the interface unit 200 to the base unit 110 as will be described in more detail below. In some embodiments, power will only be provided in one direction—i.e., from the interface unit 200 to the base unit 110 and not in the reverse direction during operation. A good reason for this approach is that the defibrillator is the most important component from a safety standpoint, and it is often undesirable to draw power from the base unit to power other devices (including the interface unit 200) in a manner that could reduce the energy available to charge the discharge capacitor in the event of an emergency. However, in some embodiments, the power supply may be bi-directional (at least in some circumstance) if desired-as for example if the base unit is not in use, is fully charged and plugged into an external charging power supply, etc.; or if the power passed to the interface unit is not coming from the base unit's internal battery (e.g., it is coming from a charger, a mobile communication device, or other device connected or attached to the base unit), etc.

The connectors 190 and 240 can take a variety of forms. They can be connectors with accompanying transceivers configured to handle processor level communications (such as UART, SPI, or I2C transceivers), with additional pins for power delivery (Power+GND), and connection verification (i.e. a pin that detects when there is a connection between the interface unit and the Base AED and triggers an interrupt on the Base AED signifying that there is not a unit connected). They can also be more standardized connectors such as USB connectors.

The interface power storage unit 250 provides power to operate the interface unit 210. In many embodiments, the power storage unit takes the form of a battery 252 with associated control components, although again a variety of other power storage technologies such as supercapacitors, ultracapacitors, etc. may be used in other embodiments. The associated control components may include components such as a battery charger and maintainer 254, which may include various safety monitors, and battery regulator 256. Preferably, the power storage unit 250 is rechargeable, although that is not a requirement. In some embodiments, the power storage unit 250 may also be arranged to supply supplemental power to the base unit 110. Depending on the structure and/or state of the base unit, the supplemental power can be used to help charge the discharge capacitor 150 during use; to power or provide supplemental power for the defibrillator electronics and/or to charge the base defibrillator unit's power storage unit 1170. In other embodiments, a supplemental battery within the interface unit (not shown) may be used to provide the supplemental power for the base unit rather than the power storage unit 250.

The location sensing module may incorporate a variety of technologies including Global Navigation Satellite Systems (GNSS) (e.g., GPS), Wi-Fi positioning, cellular triangulation, assisted GPS, Bluetooth Beacons, Near-Field Communications (NFC) and/or other location determining technologies. When requested, the interface unit can report its current location based on the location sensing technology that is believed to have the best accuracy under the then-present circumstances.

The interface unit may also optionally include various environmental sensors 263 and other peripheral components 266. When desired, the interface unit may include any of a wide variety of different types of sensors and peripheral components. For example, in selected embodiments, the interface unit may include one or more accelerometers and/or gyroscopes, a temperature sensor, a humidity sensor, a time of day or any other desired sensors or components.

The interface unit 200 is preferably configured to securely mechanically attach to the base unit 110. Typically, the interface unit is detachable such that it may be separated from the base unit if desired-although in other embodiments, the attachment may be more permanent in nature. The specific mechanical attachment utilized may vary widely in accordance with the needs of any particular embodiment. In some embodiments, press or form fitting attachment structures are used, while in others, latch and catch mechanisms, snap fit structures, etc. are utilized alone or in combination to releasably attach the interface unit to the base. However, it should be appreciated that a wide variety of other structures can be used in other embodiments. In some embodiments, the interface unit includes an attachment sensor (not shown) that senses when the interface unit is attached to a base unit.

In some embodiments, the interface unit may also include one or more biometric sensors 270. The biometric sensors may vary based on the needs of any particular defibrillator. Some of the biometric sensors may be suitable for use in detecting or evaluating CPR performed during emergency use of the defibrillator. Other biometrics may be useful in more general health management applications. For example, in some embodiments, the biometric sensors may include one or more of a pulse or heart rhythm sensor, a blood pressure sensor, a glucose monitor, a pulse oximeter, an ECG monitor, a sleep tracker, a thermometer, etc.

A benefit of the described modular defibrillator architecture is that the interface unit can be (and preferably is) designed to provide robust connectivity, effectively making the defibrillator a highly connected device. The relatively large touch sensitive display screen provides an interface that can be easily used by users of most any age. The dedicated interface unit processor(s) and corresponding memory allow the interface unit to be programmed to provide a number of functionalities that are not available in defibrillators that are commercially available today. Notably, given that the interface unit is a connected device that has a powerful processor (or processors) and a number of familiar I/O components including, for example, a touch sensitive display screen, Wi-Fi, cellular and other wireless communications capabilities, a speaker, a microphone and optionally a camera, the interface unit can be programmed to provide a number of useful functionalities without impacting the functionality of the base defibrillator unit in any way. Thus, it should be apparent that the described modular architecture helps overcome a number of practical challenges that have hindered widespread adoption of defibrillator connectivity.

### Signaling

In some implementations it is desirable for the dongle 420 or the power bank 300 itself to communicate selected information to the AED 100 (sometimes, more specifically, to the interface unit 200 of the AED) to which it is connected. For example, it can be useful for the AED/interface unit to know when it is drawing power from a supplemental power pack as opposed to wall power. Similarly, if/when multiple different types of power packs or power pack versions are available, knowing which specific power pack type/version is connected may be desirable. This type of information can be useful because the AED/interface unit may be programmed to behave differently or charge differently based on the nature of a connected power supply. For example, when the AED/interface unit knows it is being recharged by a power pack as opposed to being connected to wall power, it may be more conservative in its power usage-as for example, by checking in with, or connecting to a remotely located management server less often, or by reducing use of its display when not being actively engaged by a user, etc.

In another example, it may be desirable for the power pack or dongle to report various status information such as the power pack's charge level, operating temperature, or other operational data. The AED can either use such information for its own purposes, or forward the AED to a management server to help with AED management functions (e.g., informing remotely located administrators of the status of AEDs for which they are responsible).

There are several ways that the dongle 420 (or power pack 300) may communicate its nature and/or other desired information to a connected AED. In some implementations, the connector between the dongle/power pack and the AED/interface unit may take the form of a cable/connector with one or more data lines. For example, many USB-C cables/connectors and a variety of other cables/connectors have such data lines. In such cases, a processor on the dongle (e.g., power bank control circuitry 432 or dongle control unit 433) or on the power pack (e.g., PMIC 308) may communicate directly with a processor on the AED (e.g., interface control processor 210 or defibrillator controller 130 depending upon what component the cable connects to) to communicate the desired information. In other embodiments, the dongle 420 or power pack 300 may be outfitted with a wireless transceiver-not shown-capable of communicating with a corresponding wireless transceiver on the AED. This could be a Bluetooth module, a Wi-Fi module or any other suitable short range communication module. When such capabilities are provided, wireless communications can be used to convey the desired self-identification and/or status information.

Although data wired and wireless communications work well for the intended purposes, many charging cables (including various USB-C charging cables and many other modern charging cables) do not include dedicated data lines. When such cables are used or supported, other signaling pathways may be employed. In some embodiment, the dongle controller 433 is configured to modulate the power provided to the AED in a pre-determined sequence to signal to the AED that it is connected to a battery-based power pack as opposed to wall power. For example, the dongle may be configured to toggle the power (i.e., switch power switch 442 off and on) in a particular signaling sequence/pattern at an appropriate time so that power a predetermined sequence of timed pulses X (e.g., 2-10 pulses) at a particular frequency. One of the processors on the AED (e.g., interface control processor, 210 or defibrillator controller 130, etc.) detects the signaling sequence and, based thereon, determines that the coupled power supply is a power pack as opposed to wall power, which would not generate such a signaling sequence of pulses.

The signaling sequence can be conveyed at any desired time. For example, in some embodiments, the signaling sequence may be conveyed at the end of a charging session. In others, the signaling sequence may be conveyed at the beginning of the charging session or at a predetermined time in a charging sequence, or at two or more times during a charging session (e.g., at the beginning and at the end). Regardless of when the signaling sequence is sent/detected, once a designated signaling sequence is detected by the AED, the AED becomes aware that it is connected to a battery pack rather than wall power and can act accordingly (e.g., by implementing selected power savings techniques).

It should be apparent that when more than one type of power pack is contemplated to be used in conjunction with an AED, different types (or versions) of power packs may be arranged to use different signaling sequences so that the AED can distinguish the type of power pack to which it is connected and act accordingly. Of course, the AED can be further configured to interpret the absence of the detection of the designated signaling sequence as indicating that the AED is now connected to wall power rather than the power pack. For example, if the AED does not detect a signaling sequence at any time during a series of one or more charging cycles (e.g., after 3 charging cycles in which no signaling sequence is detected), then the AED may conclude that it is now connected to wall power and act accordingly. In the description above, frequent mention is made of the AED making certain determinations. It should be appreciated that such determinations are typically made by software (programmed instructions) executing on an appropriate processor on the AED (e.g., interface control processor, 210 or defibrillator controller 130, etc.).

In the primary described signaling embodiments, simple on/off signaling is provided. In such embodiments, the frequency, pulse width, duty cycle, off timing, number of pulses or other characteristics of the pulsing may be varied in any desired and detectable way. In other embodiments, the amplitude or shape of the pulses may be varied as part of the sequence as well. In still other embodiments, different signaling sequences can be used to convey other types of information such as power pack charge level, temperature levels, etc.

Although only a few embodiments of the invention have been described in detail, it should be appreciated that the invention may be implemented in many other forms without departing from the spirit or scope of the invention. In the descriptions above, specific power control electronics and control processes for managing power supplied by a supplemental power bank are described. However, it should be apparent that the described functionalities may be implemented using a variety of different control electronics or may be implemented in large part via one or more programmed processors that execute programmed instructions stored in memory associated with the processor(s).

In the descriptions above, representative defibrillator and interface unit architectures have been described. However, it should be appreciated that the described supplemental power banks, dongles and/or recharging system can be utilized with a wide variety of different defibrillators and/or a variety of medical devices beyond defibrillators. Similarly, the described parasitic charging of the dongle battery can be used in any battery powered dongle application.

Several flow charts have been provided to help describe some of the major functions provided by the systems. It should be appreciated that to the extent that performance isn't compromised, the order of some of the steps may be rearranged and/or some of the steps may be performed substantially concurrently. Similarly, some described steps can be combined or eliminated, while others may be added. Therefore, the present embodiments should be considered illustrative and not restrictive, and the invention is not to be limited to the details given herein, but may be modified within the scope and equivalents of the appended claims.

What is claimed is:

1. An automated external defibrillator system comprising:
an automated external defibrillator (AED) suitable for delivering a defibrillation shock to a victim experiencing a cardiac arrest, the AED including at least one rechargeable battery, a power input interface, and battery charging circuitry suitable for charging the at least one rechargeable battery using power received through the power input interface;
a supplemental power bank; and
a power delivery accessory for controlling power delivery from the supplemental power bank to the AED's power input interface, wherein the power delivery accessory includes a power management controller configured to cause the power delivery accessory to (i) periodically automatically enter an active mode thereby making power available from the supplemental power bank to the AED's power input interface, and (ii) thereafter automatically enter a deactivated mode such that power is not available to the AED's power input interface until the active mode is reentered.

2. An AED system as recited in claim 1 wherein the power delivery accessory's power management controller is configured to:
directly or indirectly monitor a current supplied to the AED's power input interface while the power delivery device is in the active mode; and
deactivate the supplemental power bank based at least in part on a determination that the supplied current does not exceed a designated threshold.

3. An AED system as recited in claim 1 wherein the power delivery accessory's power management controller is configured to activate the power delivery accessory no more often than once a day.

4. An AED system as recited in claim 1 wherein the power delivery accessory is a dongle that includes a first connector for receiving power from the supplemental power supply and a charging interface for delivering power to the AED's power input interface, wherein the charging interface is selected from the group consisting of a second connector, a USB cable, a power cable and a wireless charger.

5. An AED system as recited in claim 1 wherein the power delivery accessory further comprises an output switch controlled by the power delivery accessory's power management controller, wherein when the output switch is turned on, power is made available to the AED's power input interface, and when the output switch is turned off, power is not available to the AED's power input interface.

6. An AED system as recited in claim 1 wherein the power delivery accessory's power management controller is configured to cause the supplemental power bank to be active less than 5% of the time.

7. An AED system as recited in claim 6 wherein the power delivery accessory's power management controller is configured to cause the supplemental power bank to be active less than 1% of the time.

8. An AED system as recited in claim 1 wherein the power delivery accessory's power management controller is configured to enter the active mode after a designated period of time has passed.

9. An AED system as recited in claim 1 wherein the power delivery accessory's power management controller is further configured to:
detect new connections of the power delivery accessory to the AED and new connections of the power delivery accessory to the supplemental power bank; and
to cause the power delivery accessory to enter the active mode when a new connection is detected that results in the power delivery accessory being connected to both the AED and the supplemental power bank.

10. An AED system as recited in claim 1 wherein the supplemental power bank has an output connector through which power suitable for charging the AED is made available to the power delivery accessory, and the power delivery accessory includes a disconnect spoofing circuit directed by the power management controller that:
when set to a first state, causes the supplemental power bank to deactivate such that power suitable for charging the AED is not made available to the power delivery accessory through the output connector; and
when set to a second state, causes the supplemental power bank to activate such that power suitable for charging the AED is made available to the power delivery accessory through the output connector.

11. An AED system as recited in claim 1 wherein:
the output connector of the supplemental power bank includes at least one power pin and at least one control pin, wherein the power suitable for charging the AED is made available via the power pin;
the power delivery accessory includes a parasitic charging circuit and an energy storage device suitable for powering the power delivery accessory; and
wherein the parasitic charging circuit utilizes power from the control pin to parasitically charge the energy storage device of the power delivery accessory.

12. An AED system as recited in claim 11 wherein the energy storage device of the power delivery accessory is parasitically charged while the supplemental power bank is deactivated.

13. An AED system as recited in claim 11 wherein the energy storage device of the power delivery accessory is parasitically charged while the supplemental power bank is activated.

14. An AED system as recited in claim 10 wherein the output connector is a USB connector, and the control pin is a first configuration control pin of the USB connector.

15. An AED system as recited in claim 1 further comprising a carrying case configured to store the AED, the power accessory and the supplemental power bank, whereby the carrying case facilitates charging the AED while stored.

16. An AED system as recited in claim 15 wherein the carrying case includes an AED support insert having a bracket that holds the AED and the supplemental power bank is positioned below the AED support insert on the opposite side of the AED support insert as the AED.

17. An AED system as recited in claim 16 wherein the carrying case includes a window that exposes a display screen of the AED such that the display screen is externally visible when the AED is stored in the carrying case.

18. An AED system as recited in claim 16 wherein the carrying case further comprises a light pipe configured to make light from an indicator light on the AED more visible from outside of the carrying case when the AED is stored in the carrying case.

19. A device coupled to a power bank having a first connector that includes a first power pin and a first control pin, wherein the power bank has an active state and a deactivated state and wherein when the power bank is in the active state, power is made available via the power pin, and when the power bank is in the deactivated state, power is not made available via the power pin, the device comprising:
a second connector that couples to the first connector of the power bank, the second connector including a second power pin that connects to first power pin and a second control pin that connects to the first control pin;
an energy storage unit; and
a parasitic charging circuit, wherein when the power bank is in the deactivated state, the parasitic charging circuit utilizes power received via the second control pin to parasitically charge the energy storage unit of the device.

20. A device as recited in claim 19 wherein the energy storage device of the power delivery accessory is also parasitically charged while the supplemental power bank is in the active state.

21. A device as recited in claim 19 wherein the first and second connectors are USB connectors, and the first and second control pins are configuration control pins of the respective first and second USB connectors.

22. A device as recited in claim 19 wherein the device further comprising a disconnect spoofing circuit, wherein:
when the device is connected to the power bank, setting the disconnect spoofing circuit to a first state causes the power bank to deactivate such that power is not made available to device through the first and second power pins; and
when the device is connected to the power bank, setting the disconnect spoofing circuit to a second state causes the power bank to activate such that power is made available to the device through the first and second power pins.

23. An automated external defibrillator system comprising:
an automated external defibrillator (AED) suitable for delivering a defibrillation shock to a victim experiencing a cardiac arrest, the AED including at least one rechargeable battery and a power input interface; and
a supplemental power bank including a battery and a power management controller, wherein the supplemental power bank's power management controller is configured to (i) periodically automatically activate the supplemental power bank to thereby make power available from the supplemental power bank whereby power from the supplemental power bank is made available to the AED's power input interface, and (ii) automatically deactivate the supplemental power bank such that power is not available from the supplemental power bank until the supplemental power bank is next reactivated.

24. An AED system as recited in claim 23 wherein the supplemental power bank's power management controller is configured to activate the supplemental power bank after a designated period of time has passed.

25. An AED system as recited in claim 23 wherein the supplemental power bank's power management controller is configured to activate the supplemental power bank no more often than once a day.

26. An AED system as recited in claim 23 wherein power from the supplemental power bank is made available to the AED via a power delivery accessory selected from the group consisting of a USB cable, a power cable, and a wireless charger.

27. An AED system as recited in claim 23 wherein the supplemental power bank further comprises an output switch controlled by the supplemental power bank's power management controller, wherein when the output switch is turned on, power is made available from the supplemental power bank whereby the supplemental power bank is active, and when the outputs switch is turned off, power is not made available from the supplemental power bank whereby the supplemental power bank is deactivated.

28. An AED system as recited in claim 23 wherein the power management controller is configured to cause the supplemental power bank to be activated less than 5% of the time.

29. An AED system as recited in claim 28 wherein the power management controller is configured to cause the supplemental power bank to be active less than 1% of the time.

30. An AED system as recited in claim 23 wherein the AED further comprises battery charging circuitry suitable for controlling charging of the at least one rechargeable battery using power received through the power input interface.

31. An automated external defibrillator system comprising:
an automated external defibrillator (AED) suitable for delivering a defibrillation shock to a victim experiencing a cardiac arrest, the AED including at least one rechargeable battery and a power input interface;
a supplemental power bank; and
a power delivery controller for controlling power delivery from the supplemental power bank to the AED, wherein the power delivery controller is configured to at least sometimes toggle the power delivered to the AED in a predetermined sequence to identify a type of the supplemental power bank to the AED.

32. An automated external defibrillator system as recited in claim 31 wherein the power delivery controller is in one selected from the group consisting of:
the supplemental power bank; and
a power delivery accessory electrically connected between the supplemental power bank and the AED.
